(19) United States
(12) Patent Application Publication
(10) Pub. No.: US 2013/0178745 A1
(43) Pub. Date: Jul. 11, 2013

(54) ULTRASOUND TRACKING ADAPTER

(71) Applicant: PATHFINDER THERAPEUTICS, INC., Nashville, TN (US)

(72) Inventors: ROBERT LYNN KYLE, JR., MURFREESBORO, TN (US); BENJAMIN WILLIAM NEESE, BRENTWOOD, TN (US)

(73) Assignee: PATHFINDER THERAPEUTICS, INC., NASHVILLE, TN (US)

(21) Appl. No.: 13/624,221

(22) Filed: Sep. 21, 2012

Related U.S. Application Data
(60) Provisional application No. 61/537,691, filed on Sep. 22, 2011.

(51) Int. Cl. A61B 8/00; A61B 8/12

(52) U.S. Cl. CPC A61B 8/4263; A61B 8/4483; A61B 8/4444; A61B 8/12
USPC 600/462; 600/459

(57) ABSTRACT

An ultrasound tracking adapter assembly that attaches to an ultrasound transducer in a repeatable, rigid, and tool-less manner. When used with a tracked body, the position of the tracking technology devices and the ultrasound transducer is fixed, so intraoperative calibration is not necessary. This permits a 3D guidance system and 2D ultrasound to be used together. The adapter provides a secure, rigid hold between the ultrasound transducer body and the clamping body. The ultrasound probe clamp assembly is attached to specific probe instruments used in the operating room that are to be tracked using 3D positioning technology.

This application claims benefit of and priority to U.S. Provisional Application No. 61/537,691, filed Sep. 22, 2011, by Rob Kyle, and is entitled to that filing date for priority. The specification, figures and complete disclosure of U.S. Provisional Application No. 61/537,691 are incorporated herein by specific reference for all purposes.

### FIELD OF INVENTION

This invention relates generally to a tool for a medical tool tracking system.

### BACKGROUND OF THE INVENTION

A variety of surgical navigation systems involving the tracking of the position of medical instruments or tools during surgical procedures are well known in the art. Examples of such systems, and related tools, are disclosed in U.S. Pat. Nos. 6,190,395 and 7,043,961, the specifications, drawings, and complete disclosures of which are incorporated herein by specific reference for all purposes.

However, current systems have a number of limitations or problems, such as requiring a tool or instrument to be held at a certain angle or orientation, or establishing a clear line of sight between the camera and a certain number of emitters and/or reflectors on the position tracking device attached to the tool or instrument.

### SUMMARY

In various embodiments, the present invention comprises an ultrasound tracking adapter assembly that attaches to an ultrasound transducer in a repeatable, rigid, and tool-less manner. When used with a tracked body, the position of the tracking technology devices and the ultrasound transducer is fixed, so intraoperative calibration is not necessary. This permits a 3D guidance system and 2D ultrasound to be used together. In addition, the guidance information may be used to help the surgeon located structures of interest with the ultrasound. Further, the design of the assembly for individual transducers allows the surgeon to manipulate a transducer in hard-to-reach spaces.

The adapter provides a secure, rigid hold between the ultrasound transducer body and the clamping body. The ultrasound tracking adapter (or probe clamp) assembly is attached to specific probe instruments used in the operating room that are to be tracked using 3D positioning technology.

In one embodiment, the assembly is attached to an Aloka “T” probe (transducer body). The assembly comprises a male component and a holder or female clamp component. The male component comprises a T-end transducer body interface that fits on top of the T-shaped transducer body, a long handle (which may be low-profile, and may be curved), with a tracked portion or component at the end opposite the T-end.

The tracked portion comprises a plurality of straight or curved arms, or both. The tracked portion may be removably attached to the handle. Any number of arms may be used. A plurality of visible or infrared light sources, emitters or reflectors are placed on the arms and possibly in the center in various locations. Curved arms may allow the tool or instrument to which it is attached a greater degree of freedom of movement than is possible with prior art devices, as the light sources, emitters or reflectors can be seen from a variety of angles relative to the tracking camera or instrument. The use of arms reduces the overall weight of the adapter, and lessens interference with use of the tool or instrument.

The transducer interface of the male component is attached to the T-shaped ultrasound transducer body by placing the T-end of the male component on top of the transducer body, and then fitting the female clamp component over or around the T-end and the transducer body, in whole or in part, creating a friction mating between the components. In one embodiment, the female clamp component has an open top (formed with two side arms), with a closed bottom with an indentation suited for the transducer body. Different shapes and configurations of the female clamp component may be used with different shapes and configurations of transducer bodies, as needed.

In another embodiment, a tracking adapter may be used with an ultrasound finger probe. In this embodiment, the tracking adapter comprises a dovetail slot interface at the transducer interface end (to receive and mate with the ultrasound finger probe), a long handle section, and a tracked tool portion. The tracked tool portion may be removably attached to the end of the handle opposite the dovetail slot interface, thus allowing different forms of tracked tool sections to be used with the adapter. The dovetail slot interface snaps securely to the finger probe transducer body, and can be detached.

In this embodiment, the adapter handle may be used as a handle for the ultrasound probe, and is particularly useful for positioning the probe within a body cavity. This allows for increased space to manipulate the probe and better positioning of probe instruments on human organs, such as the liver, kidneys or pancreas.

Further, the design can be customized to individual ultrasound transducers, with clamps designed for specific probe instruments as well as a family of probe instruments. The adapter thus provides a rigid body clamp for tracking and for use as a handle, with unique geometry. It can be applied, constructed, and disassembled without the use of any tools.

### BRIEF DESCRIPTION OF THE DRAWINGS

FIG. 1 shows a perspective view of an ultrasound tracking adapter device in use with a T-probe in accordance with an embodiment of the present invention.

FIG. 2 shows views of a male clamp component of the tracking adapter of FIG. 1.

FIG. 3 shows views of a female clamp component of the tracking adapter of FIG. 1.

FIG. 4 shows a perspective view of an ultrasound tracking adapter device in use with an ultrasound finger probe in accordance with an embodiment of the present invention.

FIG. 5 shows views of the tracking adapter of FIG. 4.

### DETAILED DESCRIPTION OF EXEMPLARY EMBODIMENTS

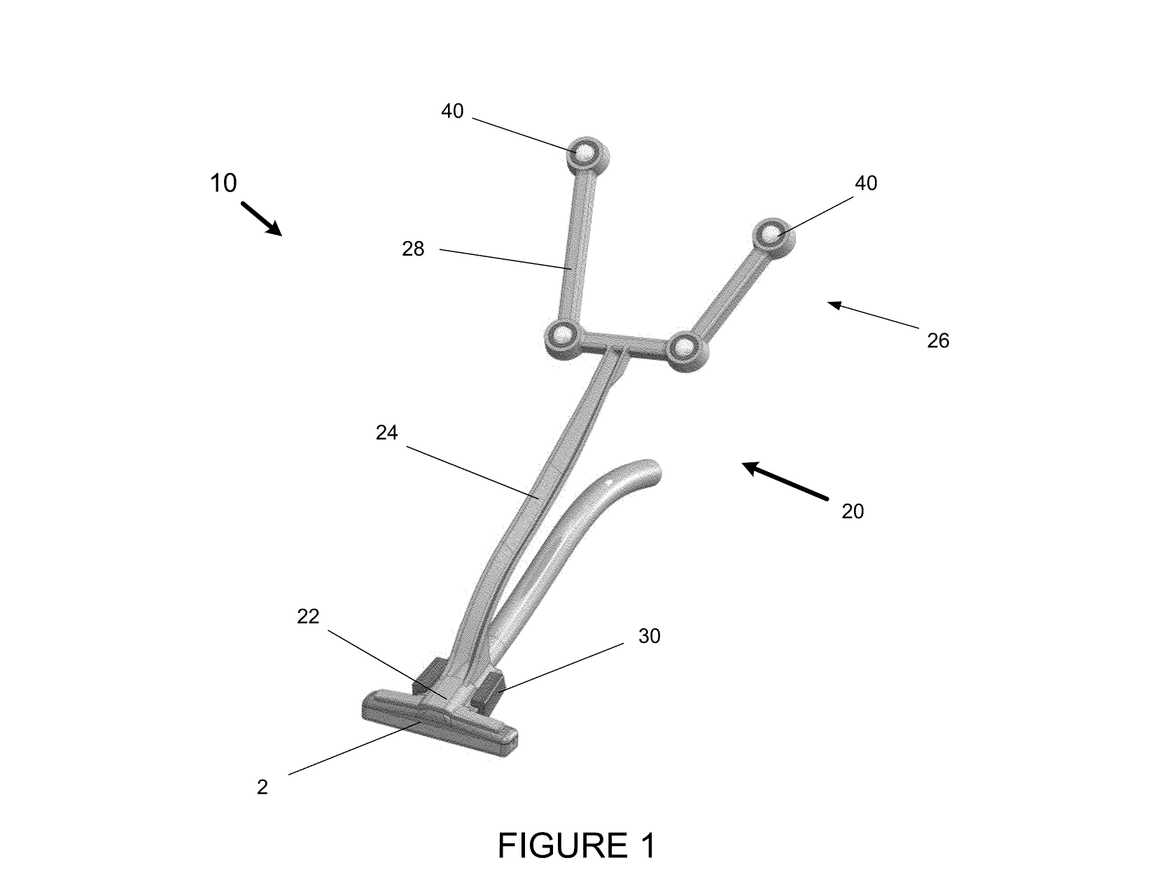

As seen in FIGS. 1-5, the present invention comprises an ultrasound tracking adapter assembly 10. The adapter attaches to an ultrasound transducer in a repeatable, rigid, and tool-less manner. When used with a tracked body, the position of the tracking technology devices and the ultrasound transducer is fixed, so intraoperative calibration is not necessary. This permits a 3D guidance system and 2D ultrasound to be used together.

The adapter provides a secure, rigid hold between the ultrasound transducer body and the clamping body. The ultrasound tracking adapter (or probe clamp) assembly 10 is attached to specific probe instruments used in the operating room that are to be tracked using 3D positioning technology.

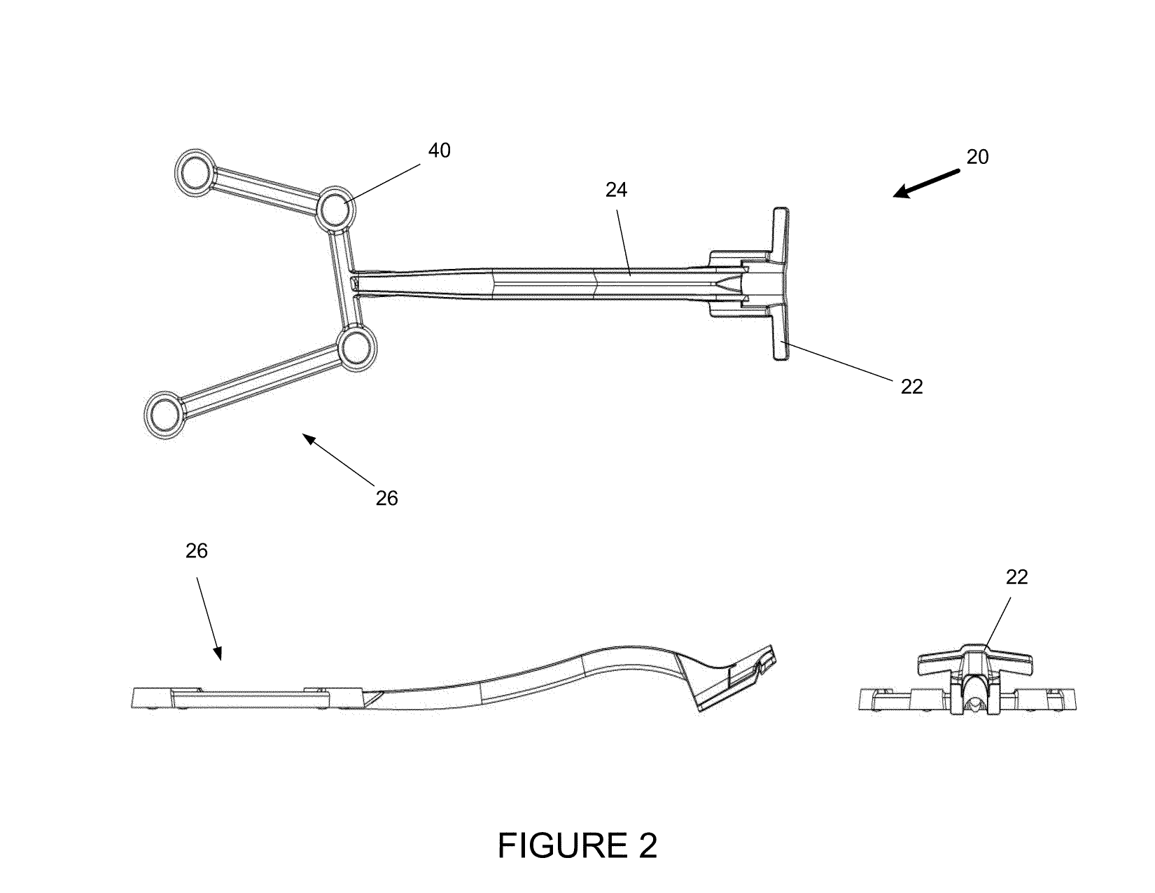

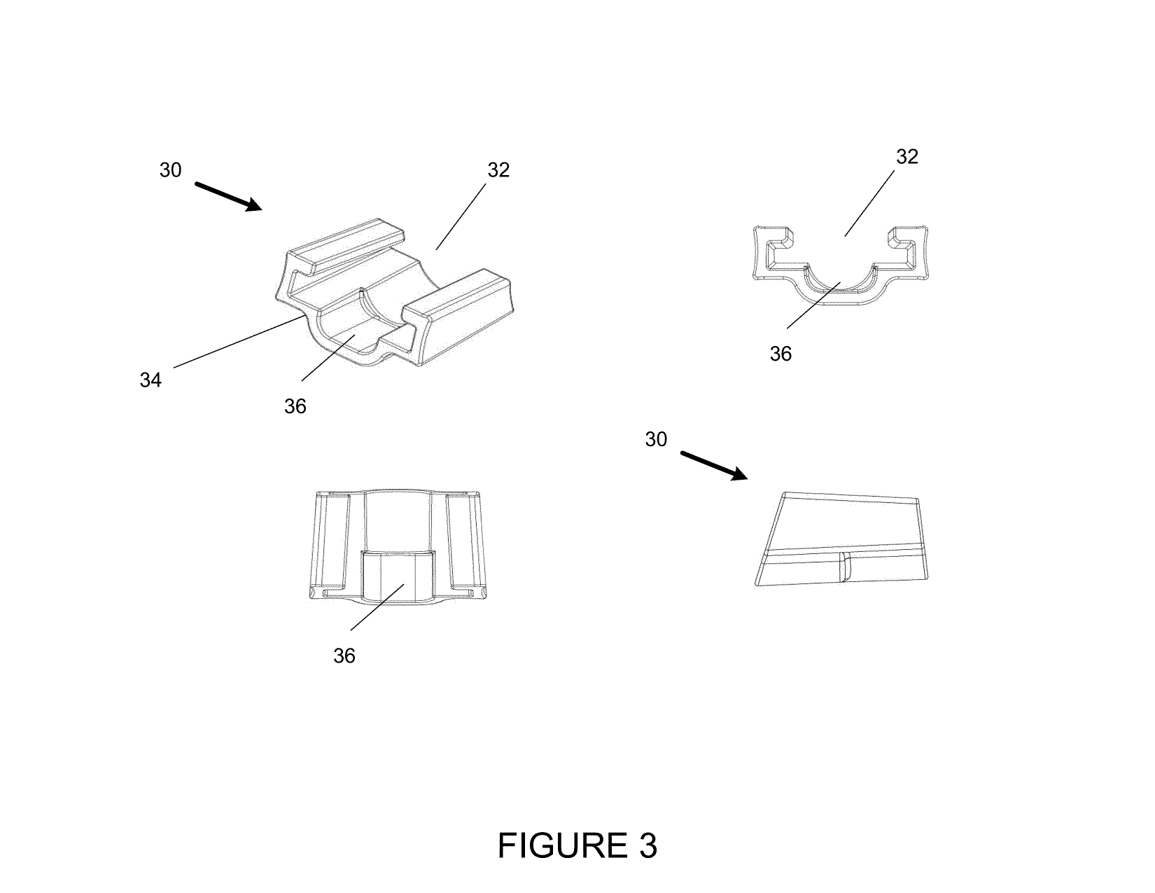

In the embodiment seen in FIG. 1, the assembly 10 is attached to an Aloka “T” probe (transducer body) 2. The assembly comprises a male component 20 (FIG. 2) and a holder or female clamp component 30 (FIG. 3). The male component comprises a T-end transducer body interface 22 that fits on top of the T-shaped transducer body 2, a long handle 24 (which may be low-profile, and may be curved), with a tracked portion or component 26 at the end opposite the T-end.

The tracked portion 26 comprises a plurality of straight or curved arms, or both 28. The tracked portion may be removably attached to the handle. While the embodiment shown in FIG. 1 shows two arms, any number of arms may be used. A plurality of visible or infrared light sources, emitters or reflectors 40 are placed on the arms and possibly in the center in various locations. Curved arms may allow the tool or instrument to which it is attached a greater degree of freedom of movement than is possible with prior art devices, as the light sources, emitters or reflectors 40 can be seen from a variety of angles relative to the tracking camera or instrument. The use of arms reduces the overall weight of the adapter, and lessens interference with use of the tool or instrument.

The transducer interface 22 of the male component 20 is attached to the T-shaped ultrasound transducer body 2 by placing the T-end 22 of the male component on top of the transducer body 2, and then fitting the female clamp component 30 over or around the T-end and the transducer body, in whole or in part, creating a friction mating between the components. In one embodiment, as seen in FIG. 3, the female clamp component has an open top 32 (formed with two side arms), with a closed bottom 34 with an indentation suited for the transducer body 2. Different shapes and configurations of the female clamp component 30 may be used with different shapes and configurations of transducer bodies, as needed.

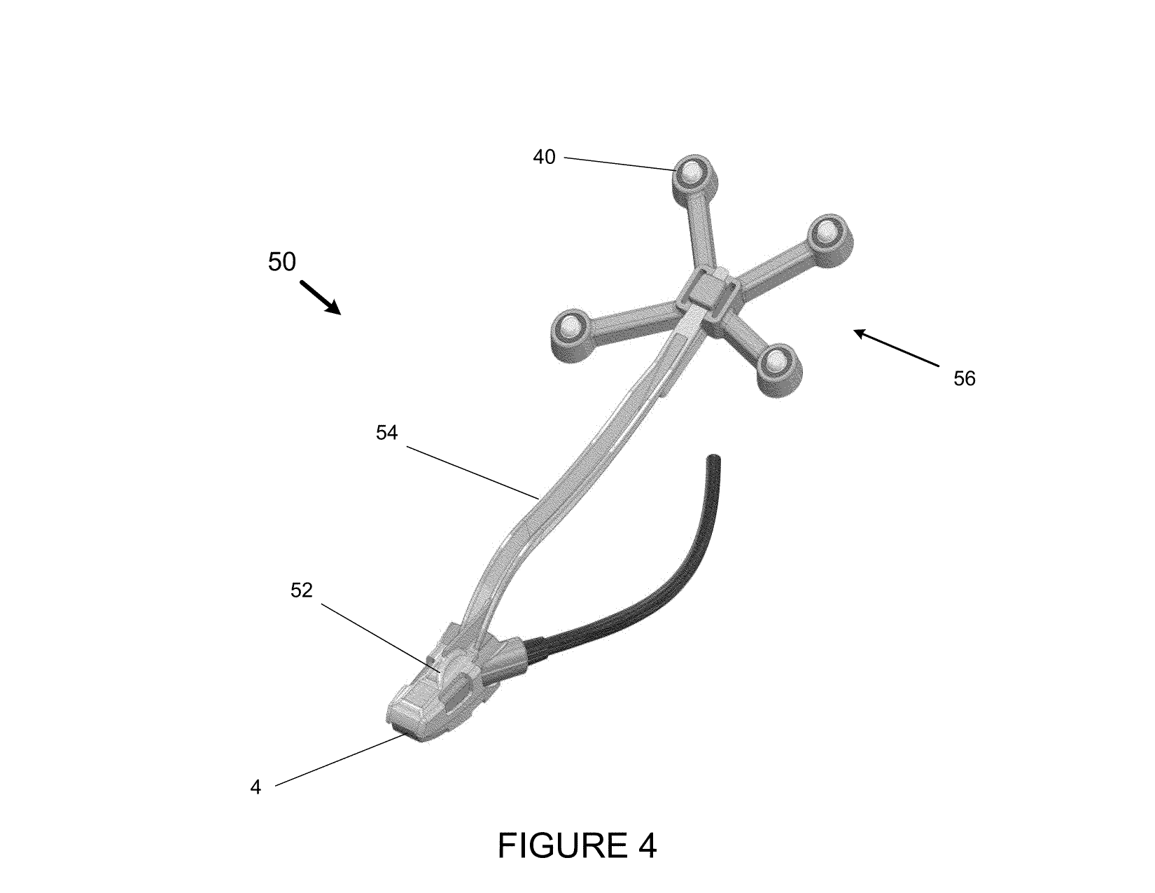

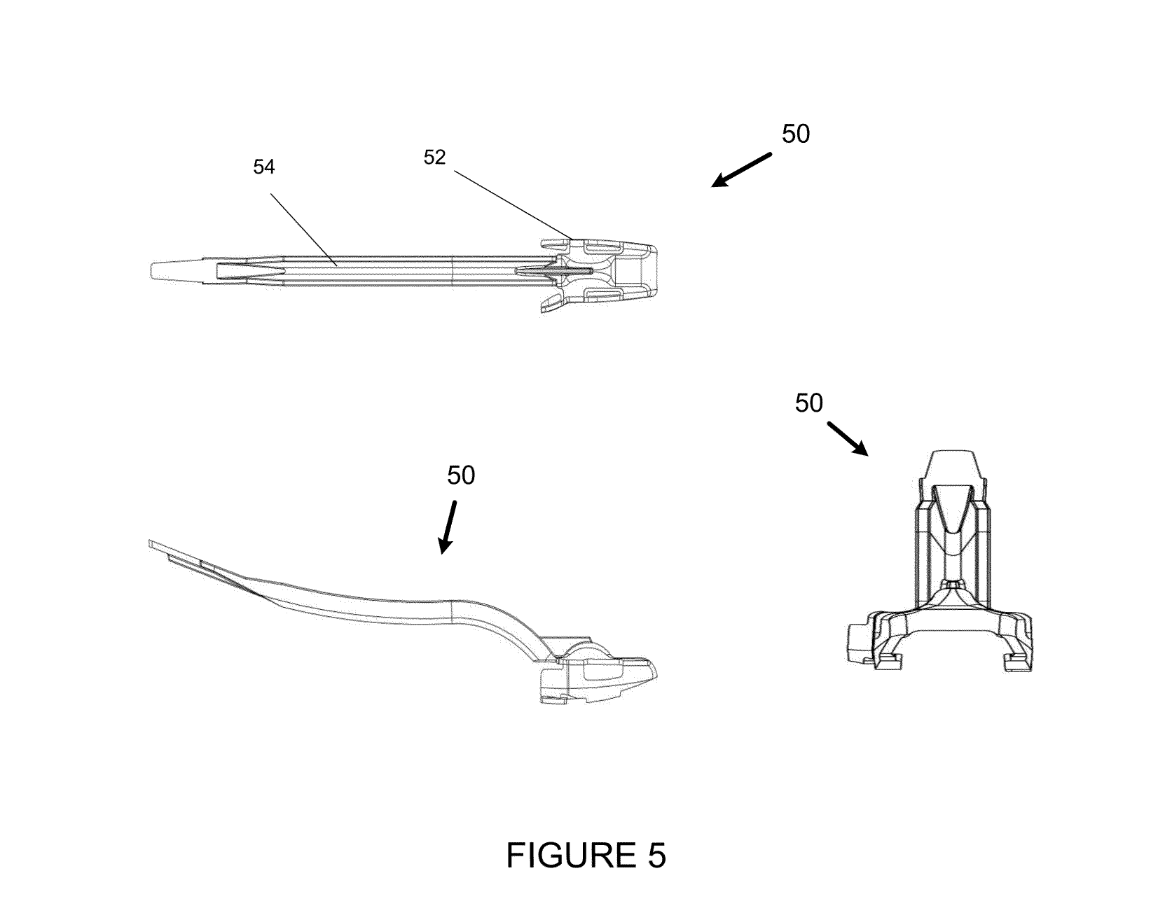

FIGS. 4 and 5 show a tracking adapter 50 used with an ultrasound finger probe 4. In this embodiment, the tracking adapter comprises a dovetail slot interface 52 at the transducer interface end (to receive and mate with the ultrasound finger probe), a long handle section 54, and a tracked tool portion 56 (as described above). The tracked tool portion in FIG. 4 is shown as removably attached to the end of the handle 54 opposite the dovetail slot interface, thus allowing different forms of tracked tool sections to be used with the adapter. In another embodiment, the device or probe may contain integrated tracking elements, thus eliminating the need for a tracked tool portion. The dovetail slot interface 52 snaps securely to the finger probe transducer body, and can be detached.

In the above embodiment, the adapter handle 54 may be used as a handle for the ultrasound probe, and is particularly useful for positioning the probe within a body cavity. This allows for increased space to manipulate the probe and better positioning of probe instruments on human organs, such as the liver, kidneys or pancreas.

Further, the design can be customized to individual ultrasound transducers, with clamps designed for specific probe instruments as well as a family of probe instruments. The adapter thus provides a rigid body clamp for tracking and for use as a handle, with unique geometry. It can be applied, constructed, and disassembled without the use of any tools.

A unique transformation to accurately map the ultrasound plane into 3D space is required for each design of the above apparatus. The transformation is calculated in a lab setting by collecting data from the devices while probing a phantom device that has known ultrasound targets as well as 3D tracking features. The collected 3D tracking data and ultrasound image data is processed and a transform is calculated. This is performed over a plurality of devices to reduce error due to manufacturing tolerances and clamping repeatability.

The calibration phantom comprises a liquid-filled vessel that the ultrasound transducer comes in contact with. Logistically, the use of such phantom in the operating room presents several problems: 1) working with an open liquid vessel creates the opportunity for the liquid to be spilled and create a hazard; 2) if the ultrasound transducer is sterile when the calibration is performed, as it would need to be for a laparoscopic procedure, the calibration phantom and liquid would both need to be sterile; and 3) it takes additional time in the operating room to perform the calibration. These problems area avoided by the method described herein.

Thus, it should be understood that the embodiments and examples described herein have been chosen and described in order to best illustrate the principles of the invention and its practical applications to thereby enable one of ordinary skill in the art to best utilize the invention in various embodiments and with various modifications as are suited for particular uses contemplated. Even though specific embodiments of this invention have been described, they are not to be taken as exhaustive. There are several variations that will be apparent to those skilled in the art.

What is claimed is:

1. An adapter device, comprising:
a rigid handle section with a first end and second end,
the first end comprising a transducer body interface section adapted to be removably connected to a transducer body, and
the second end connected to a tracked body.

2. The device of claim 1, wherein the tracked body is removably connected to the second end of the rigid handle section.

3. The device of claim 1, wherein the tracked body comprises a plurality of arms and a plurality of visible or infrared light sources, emitters or reflectors affixed to the arms.

4. The device of claim 3, wherein one or more of the arms are curved, in whole or in part.

5. The device of claim 1, wherein the transducer body is a ultrasound probe.

6. The device of claim 5, wherein the transducer body is an ultrasound finger probe, and the transducer body interface section comprises a dovetail slot interface that snaps securely to the ultrasound finger probe.

7. The device of claim 5, wherein the transducer body is an ultrasound T-probe, and the transducer body interface section is T-shaped.

8. The device of claim 1, further comprising a clamp holder adapted to removably clamp the transducer body interface section to the transducer body.
